(19) United States
(12) Patent Application Publication
(10) Pub. No.: US 2023/0310689 A1
(43) Pub. Date: Oct. 5, 2023

(54) UV LAMP COMPRISING AN AIR GUIDE FOR IMPROVING THE FUNCTION

(71) Applicant: SMART UNITED HOLDING GMBH, Gruenwald (DE)

(72) Inventor: Andreas WIESER, Muenchen (DE)

(21) Appl. No.: 18/023,152

(22) PCT Filed: Aug. 26, 2021
(86) PCT No.: PCT/EP2021/000102

§ 371 Date: Feb. 24, 2023

(30) Foreign Application Priority Data
Aug. 26, 2020 (DE) 20 2020 122 347.1

(51) Int. Cl. A61L 9/20

(52) U.S. Cl. CPC A61L 9/20; A61L 2209/134

(57) ABSTRACT

A lamp having a UV light source arranged in a housing and a light exit opening, wherein the housing has at least one further flow-through opening which is connected to the light exit opening via a flow channel, and the light exit opening connects an interior space of the housing to an exterior space in a flow-through manner.

The invention relates to a UV lamp in which an air guide is formed which, on the one hand, is provided for cooling heat-generating components in the lamp, and, on the other hand, causes a targeted air movement in the vicinity of the lamp, so that the effect of killing pathogens by the UV light is improved.

As a result of the emergence of more and more new pathogens, such as the SARS-CoV-2 virus, the measures known to date for avoiding a spread of diseases are pushed to their limits. One problem is that the development of drugs or vaccines inevitably always lags behind the emergence of new pathogens. In order to effectively prevent the occurrence of a pandemic, irrespective of precise knowledge of the pathogen or the availability of appropriate antidotes, it would therefore be desirable to be able to prevent transmission between individuals. Known systems for this purpose clean the room air, for example, by extracting the air, passing it through virus-proof filters and then releasing it back into the room. In this way, the virus load in a room (or, more generally, the load of pathogens) is actually reduced efficiently. However, the disadvantage of such systems is their high energy consumption and the fact that the virus-bearing aerosols are initially extracted. The function of such systems also suffers from movements and rapid air exchange, for example. In addition, only a general filtering of the room air takes place, so that the direct transmission of contaminated droplets between people in close proximity to each other is hardly affected.

In addition, systems are known which draw in and decontaminate air by passing the air over a path containing an area irradiated with UV light. Although such closed ventilation systems have the advantage of lower air resistance compared to the systems working with filters described above, they otherwise show the same problem, namely that only a general reduction of the pathogen density in the room air takes place. Direct transmission between people, on the other hand, cannot be prevented.

More recent considerations, on the other hand, form a narrow radiation field that can be set up like a curtain of UV light between people in a room. Since the pathogens must now pass through this radiation field when transmitted from one person to another, the pathogens can be killed by the UV light. This means that people who emit pathogens in the form of aerosol when they speak, for example, can continue to be in enclosed spaces and communicate with each other with virtually no restrictions, without the risk of transmitting diseases.

The invention now starts with such lamps that create a radiation barrier as described above. With the aid of the invention, in particular, the residence time of the pathogens in the radiation field can be increased, wherein at the same time the cooling of heat-generating components which are necessary for generating the UV radiation becomes possible.

Thus, according to the invention, a lamp is provided which has a housing in which a UV light source is arranged. The UV light parallelized by the UV light source exits (or enters) the housing via a light exit opening and extends in the direction of a main radiation direction of the UV light. In this case, the light exit opening is a flow-through region through which UV light generated inside the lamp can emerge from the housing of the lamp. In addition to this light exit opening, the housing has at least one further flow-through opening which is connected in inside the lamp via a flow channel to the light exit opening. This allows an air flow to be formed through the interior of the housing from the light exit opening to the at least one further opening or in the opposite direction. The light exit opening and the at least one further opening therefore connect the interior space of the housing with the exterior space of the housing in a manner that makes flow-through possible.

By using the light exit opening as one of the openings which separate the flow channel of the lamp from the surroundings of the lamp, the resulting air flow has a direction which has a substantial portion which is parallel to the main radiation direction of the UV light. Such a flow causes pathogens penetrating laterally into this air flow to be deflected and thus have a velocity component which extends in the direction of the main radiation direction or opposite thereto. This prolongs the residence time of the pathogens in the region in which the UV radiation is effective. In the case of unchanged dimensions of the region in which the UV radiation is effective, this means that the proportion of the killed pathogens is increased, conversely, the extent of the region of the UV radiation could be reduced without sacrificing efficacy.

Preferably, the flow channel formed in the lamp has a section leading to the light exit opening, which section is designed such that a flow direction in this section extends in the direction of a main radiation direction of the UV light emerging through the light exit opening. Such a section can be achieved, for example, by providing air guide elements in the lamp, which can also be formed by the housing itself, which extend parallel to the main radiation direction of the UV light and whose ends form the light exit opening. Ideally, no further fittings are provided in this region, so that a uniform flow in the direction of the main radiation direction or in the opposite direction can be established.

It is particularly preferred if the opening areas of the light exit opening and the at least one further opening enclose an angle of at least 45 degrees and at most 135 degrees, preferably 90 degrees. The formation of such an angle between the openings with which the interior of the housing and the surroundings of the lamp are connected has the advantage that the room air is guided in principle in a circuit passing through the lamp, whereby a residence time of the air in the region of the UV radiation which is sufficient for killing pathogens is inevitably achieved. In addition to killing pathogens that are directly transmitted between people, this also has the effect of decontaminating the rest of the room air.

Furthermore, it is preferred that a fan is provided in the housing of the lamp for generating an air flow through the light exit opening. Without the provision of such a fan, an air flow with the desired component in the opposite direction to the main radiation direction can also be formed, but this is then solely due to a convective flow. The resulting flow would thus be dependent on the temperatures occurring during operation of the lamp. If, on the other hand, a fan is used, an air flow can be formed in a targeted manner and independently of the convective flow. In particular, which is also preferred, this can be designed such that the direction of flow of the air coincides with the main radiation direction, that is to say air and light emerge from the lamp in the same direction.

With the preferred arrangement of the lamp in ceiling mounting, in addition to the radiation field which separates neighboring areas of the room from each other, an air flow is thus formed in a vertical direction downwards in the room. This vertically downward-directed flow already reduces aerosols emitted by a person on one side of the airflow from reaching the other side of the airflow for reasons of fluid mechanics alone. However, even if these aerosols enter the air flow directed vertically downward, a hazard to a person located on the other side is excluded, since possibly contained pathogens are killed by the UV light.

The UV light source has at least one illuminant and one optical device for collimating the radiation emitted by the illuminant, wherein the illuminant is arranged in the flow channel. Heat generated during the production of UV light can thus be efficiently dissipated by the air flow in the flow channel. In order to further improve the cooling, the illuminant may have a heat sink or be arranged on a heat sink, wherein the heat sink is arranged in the flow channel.

Further aspects and advantages of the lamp according to the invention will become apparent from the following description, which explains preferred exemplary embodiments of the invention with reference to the accompanying drawings. In the drawings:

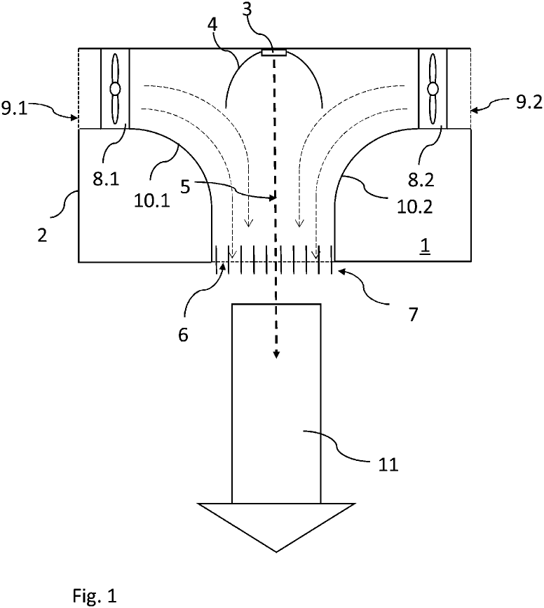

FIG. 1 shows a first section through a lamp according to the invention;

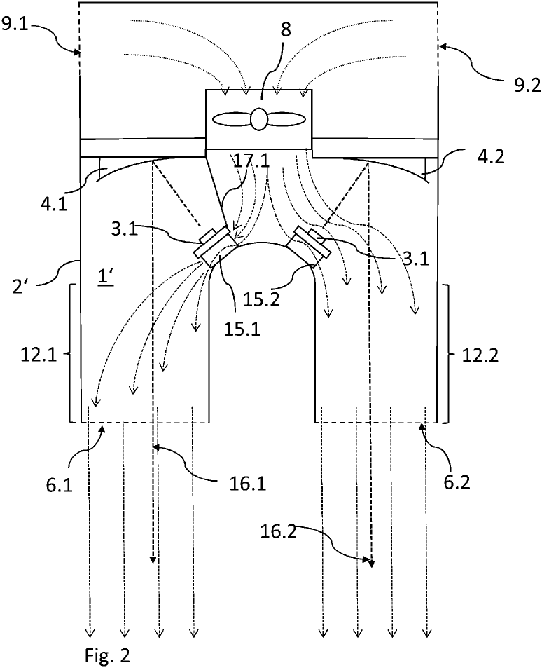

FIG. 2 shows a second example of a lamp according to the invention, with a flow channel divided into several paths;

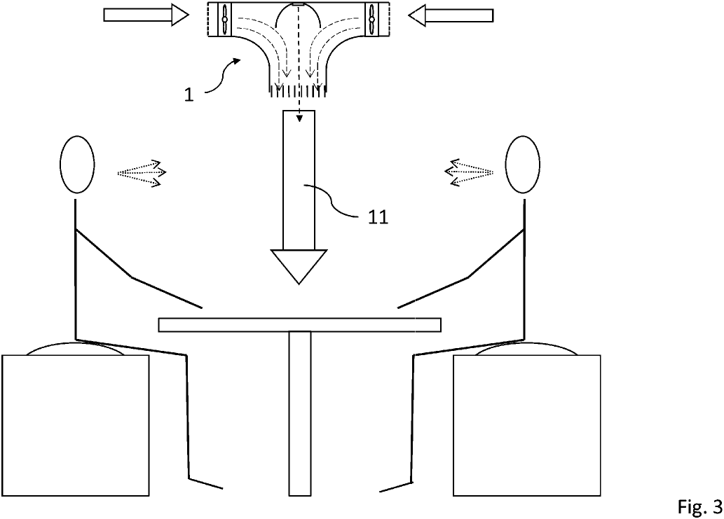

FIG. 3 shows a schematic illustration of a situation in which the lamp according to the invention is used for preventing the transmission of pathogens, and

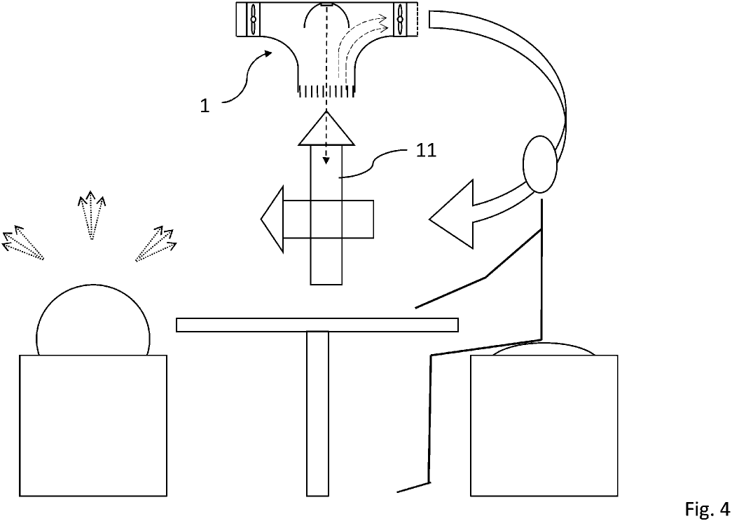

FIG. 4 shows a second illustration of a situation in which the lamp according to the invention is used for decontaminating room air.

The lamp 1 has a housing 2 in which an illuminant 3 is arranged. The illuminant 3 is an LED provided for generating UV light. The UV light emitted by the illuminant 3 is collimated by the reflector 4 so that UV radiation parallelized in the cross-section of the depicted lamp 1 emerges from the housing 2. For this purpose, a light exit opening 6 is provided in the housing 2. The light exit opening 6 is shown as a dashed line in FIG. 1 and connects the interior space of the housing 2 with the surroundings of the lamp 1 in a flow-through manner. The light exit opening 6 can be a simple opening in the housing 2. In order to fade out divergent light, an aperture system 7 can be provided in the light exit opening 6, which consists of a plurality of channels that are arranged parallel to the main radiation direction 5. In addition, this aperture system 7 may also serve for improving the directional effect of the air flow.

It should be noted that the main radiation direction 5 for the UV light is a single line only in the sectional representation of the lamp 1 shown in FIG. 1. In fact, the lamp 1 extends in a direction perpendicular to the plane of the drawing, so that the main radiation direction 5 in space is a flat area.

The housing 2 also has a first air inlet area 9.1 and a second air inlet area 9.2, wherein a first fan 8.1 and a second fan 8.2 are respectively provided on the inside of the air inlet areas 9.1, 9.2. With the aid of the fans 8.1 and 8.2, in the exemplary embodiment shown, air is drawn in from the surroundings of the housing 2, which exits again from the light exit opening 6 of the lamp 1. Thus, inside the lamp 1, a flow channel is formed which in the present example has two paths, namely, on the one hand, from the first fan 8.1 towards the light exit opening 6, and, on the other hand, from the second fan 8.2 towards the exit opening 6.

Alternatively, a fan could be provided on one side only, in which case the corresponding opposite side of the housing 2 would then be closed. It is crucial that at least one further opening is provided in addition to the light exit opening 6 such that a flow channel for an airflow is formed inside the housing 2, which allows air to flow through the housing 2. In this case, it is possible for air guide elements 10.1 or 10.2 to be provided inside the housing 2, dividing the interior space of the housing 2 in order to achieve a targeted directional effect for the air flow. It should be noted that such a directional effect can also be achieved by the housing 2 itself when the outer walls of the housing 2 appropriately limit the flow channel inside the housing 2. This will be explained below with reference to FIG. 2.

As has already been explained above, the illuminant 3 together with the reflector 4 forms a UV light source which emits parallelized UV radiation exiting the housing 2 through the light exit opening 6. The flow channel inside the housing 2 preferably has a section 12 directly adjacent to the light exit opening 6, the longitudinal extension of which is parallel to the main radiation direction 5. In this way, air drawn in through the first air inlet area 9.1 and the second air inlet area 9.2 is directed with increasing flow through the interior space of the housing 2 or the flow channel formed therein such that an air flow 11 emerging from the housing 2 through the light exit opening 6 is produced. This air flow 11 has the same direction as the emerging UV radiation.

In the preferred exemplary embodiment, as shown in FIG. 1, the air flow 11 and the UV radiation emerge from the housing 2 along the main radiation direction 5 in the same direction through the light exit opening 6. However, as has already been explained above, it is also conceivable for the light exit to be formed in the direction of the main radiation direction 5 and the air flow in the opposite direction. If necessary, additional fans 8.1 or 8.2 could then be dispensed with, provided that the flow which is convectively formed by the heat produced in the region of the illuminant 3 is sufficient.

FIG. 2 shows an alternative to the lamp 1 shown in FIG. 1. Elements corresponding to the elements of the lamp 1 already described with respect to FIG. 1 use the same reference signs and are described again only when it seems necessary for the understanding of the invention.

In contrast to the version shown in FIG. 1, the lamp 1′ now has two illuminants 3.1 and 3.2, which are each arranged on a heat sink 15.1 and 15.2. The essential beam path of the UV light emitted by the light sources 3.1 and 3.2 is illustrated by the dashed beam paths 16.1 and 16.2. The illuminants 3.1 and 3.2 are arranged in the housing 2′ such that they enclose an angle with one another and the emitted radiation moves away from one another as the distance from the illuminants 3.1 and 3.2 increases. A separate reflector 4.1, 4.2, which parallelizes the emitted UV light of the illuminants 3.1 and 3.2 respectively, is assigned to each of the illuminants 3.1 and 3.2. Similar to the example already shown in FIG. 1, the lamp 1′ also extends along its longitudinal axis into or out of the drawing plane. As in the exemplary embodiment according to FIG. 1, a plurality of light sources and associated reflectors are distributed along the length of the lamp 1 or 1′. In this case, several of these arrangements, which are successive in the longitudinal direction, can share a fan or fan pairs such that the number of fans 8 (or fan pairs 8.1, 8.2) is smaller than that of the light sources (pairs) arranged successively in the longitudinal direction of the lamp 1′.

In FIG. 2, it is shown in the right half of the lamp 1′ that only a portion of the air flow generated by the fan 8 flows through the heat sink 15.2. However, it can also be provided, and is preferred for improved cooling, that the entire air flow generated by the fan 8 flows through the heat sinks 15.1, 15.2 and the further heat sinks assigned to the same fan 8 with the aid of air guide elements. By way of example, such an air guide element 17.1 and the resulting flow for the left side of the lamp 1′ shown in FIG. 2 are shown.

In sections 12.1 and 12.2, respectively, the air flow exiting from the heat sink 15.1 is aligned such that it is substantially parallel to the emerging UV light. In this context, “substantially” means that ideally the flow direction of the air and the radiation direction of the UV light are parallel to one another over the entire cross-section of the emerging light and the airflow, which, however, cannot be completely ensured due to a divergence and turbulence in the air flow, which cannot be completely avoided, at least in the edge region.

The reflectors 4.1 and 4.2 are arranged on a common support 17, which centrally has an opening in which the individual fan 8 is arranged. On the side of the support 17 facing away from the illuminants 3.1 and 3.2 a hollow space is formed, at the lateral boundary of which through the housing 2′ the two air inlet surfaces 9.1 and 9.2 are formed. Since in this example a single fan 8 is provided for a lamp 2′ according to the invention, only one air inlet surface 9.1 or 9.2 could also be provided. With regard to the purification of room air, however, it is desirable to draw in air from both directions and to release it again through the individual fan 8 via the two light exit openings 6.1 and 6.2. As a result, a flow channel in the housing 2′ is formed by the air inlet areas 9.1 and 9.2 via the fan 8 and the two light exit openings 6.1 and 6.2. Here, the flow channel has two symmetrically running paths such that an air flow is formed out of the housing 2′ in the direction of the main radiation directions, which are shown in the present example by the beam paths 16.1 and 16.2.

As has already been explained with reference to FIG. 1, the flow direction of the air flow can also be changed in this case such that air enters the housing 2′ via the light exit openings 6.1 and/or 6.2 and, after flowing through the individual fan 8, air escapes through the further openings 9.1 and 9.2.

As can be seen directly from FIG. 2, a section 12.1 and/or 12.2 is also formed here in each case, the longitudinal extension of which corresponds in each case parallel to the beam path 16.1 or 16.2 in the region of the light exit openings 6.1 and/or 6.2. Thus, it is also achieved here that, in the sense of a nozzle, the exiting air flow is directed in the direction of the main radiation direction of the UV light. The advantages already explained with reference to FIG. 1 thus also apply to the exemplary embodiment of FIG. 2.

Since the essential direction of the airflow is effected by sections 12.1 and 12.2, which are elements of the housing 2′ itself, additional air guide elements can be dispensed with. However, it is quite conceivable that, for example, such additional guide plates nevertheless may be present for avoiding flow noise.

The space between the air inlet openings 9.1 and 9.2 can advantageously be used to accommodate, for example, the control electronics for the illuminants 3.1 and 3.2.

The above explanations only describe the cross-section through the lamp 1 or 1′. As has already been explained, the lamps 1, 1′ extend in a direction perpendicular to the drawing plane. Several of the fans 8 or the fans 8.1 and 8.2 can therefore be arranged along the length of the lamps 1, 1′, just as a plurality of illuminants and the corresponding collimating optical elements are arranged distributed along the longitudinal axis of the lamp 1, 1′.

The separate arrangement of illuminant and associated reflector shown in FIG. 2 is also applicable to the lamp 1 as shown in FIG. 1, in which only one light exit opening is provided and the reflectors are arranged centrally for concentrating the UV radiation. In any case, the separate arrangement has the advantage that the illuminants (or their heat sinks 15.1 and 15.2) can be well arranged in the flow and thus efficient cooling is achieved.

The arrangement shown in FIG. 2 shows the LEDs 3.1 and 3.2 as well as their associated reflectors 4.1 and 4.2 in a plane with LEDs 3.1 and 3.2 arranged on the inside. Alternatively, the LED 3.1 and the reflector 4.1 can also be arranged axially offset to the LED 3.2 and its reflector 4.2. Preferably, the LEDs 3.1 and 3.2 are then arranged on the outer walls of the housing 2 such that the reflectors 4.1 and 4.2 are then arranged in the center of the housing. In a cross-sectional view, the reflectors 4.1 and 4.2 then overlap.

Using the lamp as shown in FIG. 1, the use of the lamps according to the invention will now be explained with reference to FIGS. 3 and 4. For better clarity, not all components of the lamp 1 according to the invention already explained in FIG. 1 are again provided with reference signs.

In the situation shown in FIG. 3, two people are sitting across from each other, for example, when visiting a restaurant. When people speak and laugh, they emit aerosols which, if one of the two people is infected, contain pathogens. The lamp 1, 1′ according to the invention is intended to ensure that the transmission of diseases from one person to another is reliably prevented. For this purpose, the lamp 1 emits UV radiation propagating along the main radiation direction. As has already been explained above, the lamp 1 extends in a direction perpendicular to the drawing plane such that a kind of curtain of UV radiation is created, which is arranged between the two people. To transmit diseases, pathogens would now have to travel from one side of the UV radiation to the other. As shown in FIG. 3, the flow direction of the air flow exiting the lamp 1 after passing through the flow channel and the radiation direction of the UV light are identical. Any pathogens that may enter the region of the UV radiation are deflected by this air flow such that they no longer pass through the UV radiation by the shortest route. Rather, their residence time within the region of UV radiation increases, ensuring that the pathogens are killed.

In addition, it is an advantage that air is drawn in from the vicinity of the lamp 1, which may also contain pathogens. This drawn-in air is transported through the flow channel which is formed inside the lamp 1 and exits together with the UV radiation from the light exit opening. As a result, pathogens which may be present in the room air are actively brought into the region irradiated with UV light such that the lamp is also decontaminating the room air independently of the two people present.

Another example is shown in FIG. 4: Here, the flow direction is reversed such that air is drawn in through the light exit opening 6. While the left fan 8.1 is switched off and, if necessary, the first air inlet opening 9.1 is closed, the second fan 8.2 discharges air that was drawn in through the light inlet opening 6 via the second air inlet opening 9.2 (further opening). In the room as a whole, an air flow is created which protects the person sitting to the right of the table in FIG. 4 from any source of pathogens, such as is shown as an example on the left side of the table. The air flow that forms approximately forms a circuit that returns from the further opening 9.2 back to the intake area. However, this creates a flow that counteracts the spread of aerosols or pathogens in general from any source towards the person sitting on the right. In this way, a protective decontamination of room air can also be achieved in a targeted manner by selective activation in the case of several fans.

It should be noted that the precise formation of the UV radiation is irrelevant for the present invention. In addition to the example shown using LEDs as illuminant, it is also possible to use tubes for generating the UV radiation. Alternatively, lenses can also be used to achieve the parallelization of the UV light emitted by the illuminant. The invention can also be used with such lenses instead of reflectors. It is essential for the invention that a flow channel is formed inside the lamp 1, 1′ which generates an air flow which has the same direction or an opposite direction as the radiation direction of the UV light either when exiting or entering through the light exit opening 6. By using the light exit opening 6 as well as at least one further opening (air inlet area 9.1, 9.2), which are preferably approximately perpendicular to one another or enclose at least one angle between 45 and 135°, an advantageous air flow can be generated in the roo m in addition to the cooling of the heat-generating components inside the housing 2. In addition to preventing the direct transfer of pathogens from one person to another person who are separated from one another by the region irradiated with UV light, a reduction in virus or bacteria load in the room air in general can thus be achieved.

1. A lamp having a UV light source arranged in a housing and a light exit opening, wherein the housing has at least one further flow-through opening which is connected to the light exit opening via a flow channel, and the light exit opening connects an interior space of the housing to an exterior space in a flow-through manner.

2. The lamp according to claim 1, wherein the flow channel formed in the lamp has a section leading to the light exit opening, which section is designed such that a flow direction in this section extends in the direction of a main radiation direction of the UV light emerging through the light exit opening.

3. The lamp according to claim 1, wherein the opening areas of the light exit opening and the at least one further opening enclose an angle of at least 45 degrees and at most 135 degrees, preferably 90 degrees.

4. The lamp according to claim 1, wherein at least one fan is provided in the housing for generating an air flow through the light exit opening.

5. The lamp according to claim 1, wherein the UV light source has at least one illuminant and one optical device for collimating the radiation emitted by the illuminant.

6. The lamp according to claim 4, wherein the illuminant is arranged in the flow channel.

7. The lamp according to claim 5, wherein the illuminant may have a heat sink and the heat sink is arranged in the flow channel.

8. The lamp according to claim 6, wherein the illuminant may have a heat sink and the heat sink is arranged in the flow channel.
